(19) United States
(12) Patent Application Publication
(10) Pub. No.: US 2003/0215525 A1
(43) Pub. Date: Nov. 20, 2003

(54) Heat stable antacid and antigas suspensions

(76) Inventors: Douglas S. Beyerle, Lansdale, PA (US); John J. Dubek, Philadelphia, PA (US); Gerard P. McNally, Berwyn, PA (US); Donald L. Hasenmayer, North Wales, PA (US); John Case, Perkasie, PA (US); Abbie Gentry, Horsham, PA (US); Indu G. Shah, North Wales, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/304,837

(22) Filed: Nov. 26, 2002

Related U.S. Application Data
(63) Continuation-in-part of application No. 09/157,795, filed on Sep. 21, 1998, now abandoned.

(51) Int. Cl. A61K 33/12; A61K 33/08; A61K 33/10; A61K 31/717

(52) U.S. Cl. 424/683; 424/690; 424/687; 424/686; 514/57

(57) ABSTRACT

The invention relates to heat stable liquid antacid and/or antigas preparations capable of being pasteurized in the temperature range of 60-100° C. comprising one or more acid neutralizing and/or antigas compounds in an aqueous liquid suspension consisting essentially of hydroxyethylcellulose as a suspending agent.

### CROSS REFERENCE TO RELATED APLICATIONS

This application is a continuation-in-part of U.S. application Ser. No. 09/157,795 filed Sep. 21, 1998.

### BACKGROUND

1. Field of the Invention

The present invention relates to liquid antacid compositions and methods for their preparation. More particularly, the present invention relates to heat stable liquid antacid and antigas preparations containing suspending agents which are capable of being pasteurized without gelling or interacting with the antacid metal salts in the compositions.

2. Description of the Related Art

Gastric antacids are agents that neutralize or remove acid from the gastric contents. Antacids are widely used in the treatment of various gastrointestinal disorders such as peptic ulcers and gastritis. Antacids are also used for the relief of acid indigestion, heartburn, dyspepsia, sour stomach, reflux esophagitis and the like. The clinical use of antacids is based on their ability to neutralize stomach acid and increase the pH of gastric secretions.

Antacids used today are made from a variety of inorganic salts such as calcium carbonate, sodium bicarbonate, magnesium salts and aluminum salts. Magnesium hydroxide and aluminum hydroxide are the most potent magnesium and aluminum salts and are often used in combination. In addition, magnesium oxide, magnesium carbonate, aluminum phosphate, magaldrate, magnesium trisilicate, and aluminum sucrose sulfate (sucralfate) are also employed.

Antigas preparations are those used as adjuncts in the symptomatic treatment of flatulence, functional gastric bloating, and postoperative gas pains. The clinical use of such antigas preparations such as simethicone is based on their antifoam properties. Silicone antifoams spread on the surface of aqueous liquids, forming a film of low surface tension and thus causing the collapse of foam bubbles. This leads to symptomatic relief of pain and distress associated with gas.

Antacids and antigas preparations are available in both liquid suspensions as well as solid dosage forms. In general, liquid suspensions are preferred to tablets or powders since they are more rapidly and effectively solubilized and have a greater ability to react with and neutralize gastric acid or reduce gas. The major benefit of such liquid preparations are their speed of onset. However, a difficulty associated with liquid preparations in general is adequate preservation and terminal sterilization of the finished packaged product. It is key to the preservation of these products that they be packaged into their containers in as microbially clean a state as is possible (i.e. terminally sterilized). This sterilization can be accomplished by either (i) adding chemicals such as sodium hypochlorite or hydrogen peroxide to the products as they are packaged (U.K. Pat. GB 1275885) or (ii) heat treatment (pasteurization) of the liquid antacid.

These sterilization methods suffer from certain disadvantages however. A disadvantage of (i) is that a bad taste is conferred to the product and also it requires that the product be bottled before the chemicals decay which will be a function of how much is initially added. The major concern with (ii) is the effect which heat has on the components of the formulation. One particular problem is the effect of heat on the suspending agents (gums) used to maintain the antacids in suspension. The problem is that the suspending agents tend to undergo thermal gelation at the temperature ranges used in the pasteurization process. The choice of suspending agents available is already limited to those which do not interact with metal ions. Of the suspending agents available, hydroxypropyl methylcellulose (HPMC) is most commonly used and has conventionally been thought to be the best suited because of its compatibility with antacids (metal ions) and the fact that it could be pasteurized at moderate temperatures 60-70° C.

However, pasteurization in this temperature range is less than ideal. Another problem is that only limited amounts of product can pass through the pasteurizer unit before it becomes blocked with gelled HPMC. Thermal gelation is a known phenomenon associated with HPMC(see for example, A. Haque et al., Thermogelation of Methylcellulose. Part II: effect of hydroxypropyl substituents, Carbohydrate Polymers 22 (1993) 175-186). The thermal gelation of the suspending agent in the pasteurization process therefore limits the production capacity and leads to increased costs in manufacturing.

It is therefore the object of this invention to provide a liquid antacid composition which is capable of being pasteurized in the temperature range 60-100° C. without gelling or interacting with the divalent and/or trivalent antacid metal salts contained therein.

### SUMMARY OF THE INVENTION

The invention relates to heat stable liquid antacid and/or antigas preparations capable of being pasteurized in the temperature range of 60-100° C. comprising one or more acid neutralizing and/or antigas compounds in an aqueous liquid suspension containing hydroxyethylcellulose as suspending agent. It has been found that when hydroxyethylcellulose (HEC) is used as a suspending agent for antacid/antigas suspensions the resulting product has the following properties:

(i) It can be readily sterilized by pasteurization without the concerns of the suspending agent gelling in the pasteurizer. Specifically, the viscosity of the resulting product is from about 100 to 1000 cps at 25° and 70° C. This is not the case with other cellulosic gums such as HPMC,s or HPC.

(ii) In the case of aluminum, calcium or magnesium containing antacid compositions, the HEC does not interact with the trace amounts of free multivalent metal ions (such as Al³⁺, Ca²⁺, or Mg²⁺ cations) in solution as is the case with other suspending agents, e.g. xanthan gum, carrageenan, carbopol, carboxymethylcellulose sodium, alginates, locust bean gum.

(iii) The HEC does not have an inherently high bioburden as do many of the naturally occurring gums such as guar, acacia, tragacanth, locust bean gum etc.

The advantage of using HEC's as suspending agents in antacid and/or antigas suspensions (particularly those containing aluminum, magnesium or calcium containing antacids) allows for easy terminal sterilization of the product by a pasteurization process.

### DETAILED DESCRIPTION

The invention relates in particular to liquid antacid and/or antigas preparations comprising an effective amount of one or more acid neutralizing compounds and/or antigas compounds, an hydroxyethylcellulose (HEC) suspending agent, and, optionally, one or more other pharmaceutically acceptable additives. Preferably, the preparation contains 1.0 mg-50 mg/5 ml HEC suspending agent.

The antacid compounds applicable for use as the active acid neutralizing compound in the present invention are the salts of aluminum, magnesium and calcium conventionally used in liquid antacid compositions. Generally, the composition contains about 200 mg-2000 mg/5 ml acid neutralizing compound. For instance, calcium carbonate in the range of 200 to 2000 mg per 5 ml may be employed, as well as magnesium carbonate, magnesium trisilicate, aluminum hydroxide and magnesium hydroxide and mixtures thereof. The amount of antacid in the preparation may conveniently be, for example, in the range of 5% to 35% w/v of the composition. A mixture containing from about 5 to about 15% w/v calcium carbonate and about 2 to about 8% magnesium carbonate or magnesium trisilicate may advantageously be employed. Aluminum hydroxide gel in an amount comprising about 150 to about 800 mg per 5 ml may also be employed. The active acid neutralizing compounds are generally utilized as individual powders, preferably micronized powders.

Alternatively, or in addition to the acid neutralizing compound, the composition may contain an antigas compound such as simethicone as an active ingredient. Such antigas compounds are those used as an adjunct in the symptomatic treatment of flatulence, functional gastric bloating, and postoperative gas pains. For self medication in over-the-counter preparations, antigas compounds such as simethicone are used as an antiflatulent to relieve symptoms commonly referred to as gas, including upper GI bloating, pressure, fullness, or stuffed feeling. Simethicone is often combined with other gastrointestinal medications, such as antacids, antispasmodics or digestive enzymes. The simethicone preferably conforms to the United States Pharmacopoeia (USP XXII) definition, that is, a mixture of fully methylated linear siloxane polymers containing repeating units of the formula (—(CH₃)₂SiO—)ₙ, stabilized with trimethyl siloxy end-blocking units of the formula (—(CH₃)₃—SiO—) and silicon dioxide. Other organopolysiloxane antifoam agents are known in the art and may also be used as the active ingredient in this invention. Such organopolylosiloxane antifoam agents are disclosed, for example, in U.S. Pat. No. 5,458,886, and the references discussed therein, hereby incorporated by reference.

The amount of simethicone or other organopolysiloxane antifoam agent contained in the solid oral dosage form should be sufficient to provide a therapeutic dosage to a patient suffering from gas or flatulence and associated symptoms. The preferred dosage range for simethicone is in the range of about 20 mg to about 125 mg per dosage unit, generally not to exceed 500 mg/day. The dosage ranges may vary for age and weight of a patient as well as the severity of symptoms.

The suspending agent is chosen from the hydroxyethylcellulose (HEC) polymers known in the art. HEC is a water soluble polymer derived from cellulose which is nonionic and is therefore unaffected by cations in solution, unlike other ionic cellulose ether polymers. This property makes it ideally suited for use with divalent and/or trivalent cationic antacid salts commonly used. HEC is produced from cellulose by treatment with sodium hydroxide and reaction with ethylene oxide to introduce hydroxyethyl groups to yield a hydroxyethyl ether. By performing the reaction under certain conditions, the degree of substitution of hydroxyl groups can be controlled. The solubility in water is achieved as the degree of substitution is increased. HEC is available in various viscosity and solubilty types as NATROSOL® from Aqualon, a division of Hercules Incorporated, Wilmington Del., or as TYLOSE® from Hoechst Celanese Corporation. In accordance with the invention, HEC is present in the liquid composition as suspending agent in an amount ranging from 1 mg per 5 ml to about 100 mg per 5 ml or about 0.02% w/v to about 2% w/v of the composition.

The liquid antacid suspension may also advantageously contain a preservative component selected from any pharmaceutically acceptable preservative. The alkyl esters of para-hydroxybenzoic acid (the parabens, e.g. butylparaben, methylparaben and propylparaben) are preferred and may be used alone or in combination. Generally, the parabens are used in a concentration of about 0.02% w/v. Other preservatives include ethylenediamine tetra-acetic acid, propyl-p-hydroxybenzoates, sodium benzoate or sorbic acid.

In one embodiment, an antimicrobial adjuvant, selected from propylene glycol, glycerin, and mixtures thereof, is present in the preparation in a total amount ranging from about 2 to about 20%. Additionally, the preparation may also be substantially free of parabens or bis-biguanides, or other conventional preservatives.

In embodiments where propylene glycol is employed as a sole antimicrobial adjuvant, the propylene glycol is present in the preparation in an amount ranging from greater than 2 to less than 15 weight percent of the total weight of the preparation. Preferably, in these embodiments, the preparation comprises about 3 to about 11 weight percent propylene glycol, more preferably about 4 to about 6.25 weight percent propylene glycol. In embodiments where propylene glycol is employed in combination with glycerin, the amount of propylene glycol is preferably from about 3 to about 10%, e.g. from about 4 to about 7%, or in one particular embodiment, the level of propylene glycol is 5%.

Preferably up to about 20 weight percent glycerin is present in the preparation. In embodiments wherein glycerin is the sole anti-microbial adjuvant, the level of glycerin is preferably from about 15 to about 20 weight percent. In embodiments wherein glycerin is employed in combination with propylene glycol, the level of glycerin is preferably from about 3 to about 10%, e.g. from about 4 to about 7%, or in one embodiment the level of glycerin is 4%. Glycerin in particular has been found to impart good taste to the preparation.

A particularly preferred preparation according to the invention employs about 4 to about 6.25 weight percent propylene glycol and about 3 to about 7 weight percent glycerin. This combination has been found to provide excellent preservative efficacy and taste.

Advantageously, the liquid antacid preparation may also contain a histamine H2 receptor antagonists or other active agent typically used in gastrointestinal medications. Histamine H2 receptor antagonists are agents which reduce acid secretion and are effective in the treatment of many gastric disorders. Co-administration of histamine H2 receptor antagonists and an antacid is known for example from U.S. Pat. No. 5,229,137 and WO 92/00102. Any of the known histamine H2 receptor antagonists may be used such as cimetidine, ranitidine, nizatidine and famotidine. A typical preparation will contain about 100 mg to about 400 mg of cimetidine, or 50 mg to about 150 mg of ranitidine or 10 mg to 40 mg of famotidine per dosage unit (e.g., per 5 ml). Typically, the histamine H2 receptor antagonist is employed as the free base or, in the form of the physiologically acceptable salt, such as the hydrochloride salt in the case of ranitidine. Other active agents may be added to the preparation. For instance, analgesics, antidiarrheals, or antispasmodic-agents may be added as well as other gastrointestinal agents in dosage amounts conventionally used in the treatment of gastrointestinal dysfunction.

The composition according to the invention, in unit dosage form, may be administered, for example 1 to 4 times per day. The dosage will depend on the active agents that are employed, the condition being treated and the age and weight of the patient. Typical dosages include about 5-30 mls of the preparation containing the dose of antacid selected to achieve the desired acid neutralizing effect. A suitable dose range for calcium carbonate is 200 mg to 2.0 g.

The liquid compositions of the invention are aqueous suspensions containing the active ingredients in admixture with pharmaceutically acceptable excipients typically found in aqueous suspensions for oral administration. Such excipients may be dispersing or wetting agents such as sorbitan esters or lecithin, surface modifiers, aqueous or non-aqueous vehicles such as sorbitol solution, ethyl alcohol or fractionated vegetable oils, or diluents.

The compositions may also contain flavorings, colorants and/or sweeteners as appropriate. Suitable flavorants include fruit flavors, peppermint, licorice or bubble gum flavors. The sweetening agents may be for example bulk sweeteners such as sugars (e.g. sucrose or fructose) or polyols (e.g. maltitol, sorbitol) and/or intense sweeteners such as saccharin, aspartame or acesulfame K.

In addition, the composition may contain buffering agents such as tri or di-ester buffers such as triacetin, or other buffers and buffer salts such as tartaric acid or citric acid.

The liquid antacid compositions of the present invention may be prepared according to conventional techniques well known in the pharmaceutical industry. Thus, for example, the antacid, and the suspending agent may be admixed, if desired, with suitable excipients and dispersed in the aqueous vehicle.

The antacid composition of the present invention is in the form of a liquid, and is designed to be readily pourable and pumpable. Accordingly, the composition preferably has a water content of at least about 40 weight percent, e.g., from about 40 to about 70 weight percent.

As stated, the use of the HEC suspending agent in the antacid composition of the present invention provides significant advantages in the manufacturing of liquid antacid compositions requiring the use of a suspending agent because terminal sterilization can be accomplished by pasteurization without the concerns of the suspending agent gelling in the pasteurizer. This is not the case with other cellulosic gums such as HPMC,s or HPC. The HEC containing antacid composition of the present invention advantageously undergoes minimal change in viscosity upon heating, enabling it to be easily pumped through a pasteurizer at elevated temperatures. In particular, the liquid antacid composition of the invention before pasteurization has a viscosity at 25° C. of about 100 to 1000 cps, e.g., about 200 to about 600 cps. The liquid antacid composition of the invention has a viscosity at 70° C. of about 100 to about 1000 cps, e.g. about 200 to about 600 cps. The liquid antacid composition as a finished product after pasteurization has a viscosity at both 25° C. and 70° C. of about 100 to about 1000 cps, e.g., about 200 to about 600 cps. Advantages of the invention are demonstrated in the results of the following Example 1 wherein it is shown that the composition of the present invention containing HEC as suspending agent exhibited an increased flow rate in the pasteurization process over the comparative formulation containing HPMC as suspending agent.

In order to further illustrate the present invention and the advantages thereof, the following specific examples are given, it being understood that these examples are intended only to be illustrations without serving as a limitation on the scope of the present invention.

### EXAMPLE 1

Comparing the Heat Stability of HEC vs. HPMC.

A recirculation study was conducted on the Plant Scale APV Heat Exchanger. The purpose of this study was to compare the magnitude of fouling (clogging) observed in the heat exchanger with an antacid formula suspended with hydroxypropyl methylcellulose (HPMC) versus an equivalent formula suspended with hydroxyethylcellulose (HEC).

HPMC and HEC are both cellulose based hydrocolloids that enhance the viscosity of a liquid product. Table I highlights the unit formulae used for this study. It should be noted that the RT viscosity (Brookfield spindle #2 speed 12) of these formula are equivalent (225 cps)

Two 500 gallon batches were prepared for this study. After processing, both batches were homogenized at 1600 psi prior to pasteurization.

TABLE I

| | Formula 1 | Formula 2 |
|---|---|---|
| Raw Material | mg/5 ml | mg/5 ml |
| Sorbitol Solution | 953 | 953 |
| Purified Water | 3370.5 | 3381 |
| Hydroxypropyl methylcellulose K4M* | 28.028 | |
| Hydroxyethylcellulose TYLOSE 4000** | | 17.5 |
| Avicel RC 591 | 13 | 10 |
| Simethicone Emulsion (30%) | 73.11 | 73.11 |
| Magnesium Hydroxide Powder | 210.5 | 210.5 |
| Aluminum Hydroxide Gel (13%) | 787.4 | 787.4 |
| Butylparaben | 1 | 1 |
| Propylparaben | 1.5 | 1.5 |
| Creme de Menthe Flavor | 0.233 | 0.233 |
| Lemon Flavor | 18.1 | 18.1 |

*Dow Chemical
**Hoechst Celanese

Table II illustrates the reduced clogging of the heat exchanger when the present invention using HEC is compared with the equivalent current HPMC formulation. Product was recirculated for 5 minutes then manually drained at ˜1.2 gpm for 15 minutes. After draining, the product was recirculated for 5 more minutes and then drained for 15 minutes. This was repeated for 210 minutes. Changes in pump pressure and pump % were monitored throughout the process. An increase in pressure and/or pump % indicates that the heat exchanger is clogging and ultimately would need to be shutdown prematurely.

Table II highlights these results. The results show no significant change in these parameters for the HEC formulation of the present invention, while the HPMC formulation exhibited significant increases in pump pressure and pump %. Overall the results indicate that the HEC formulation shows no evidence of fouling the pasteurizers.

TABLE II

| | | | |
|---|---|---|---|
| | Pump Pressure | | |
| Gallons | (psi) | Pump % | GPM |

Formula 1 (HPMC)

| | | | |
|---|---|---|---|
| 0 | 31 | 45 | 1.2 |
| 40 | 54 | 62 | 1.2 |
| 75 | 65 | 70 | 1.2 |
| 100 | 81 | 80 | 1.2 |
| 125 | 94 | 93 | 1.2 |

Formula 2 (HEC)

| | | | |
|---|---|---|---|
| 0 | 34 | 62 | 1.2 |
| 40 | 35 | 58 | 1.2 |
| 75 | 35 | 57 | 1.2 |
| 100 | 35 | 59 | 1.2 |

In conclusion, it appears major improvements in product throughput prior to complete fouling of the heat exchanger can be achieved by replacing HPMC in an antacid formula with HEC.

### EXAMPLE 2

Formulation Containing Aluminum Hydroxide Gel.

| | Mg/5 ml |
|---|---|
| Al (OH)₃ Gel (13% Al₂O₃) | 1538.5 |
| Sorbitol (70% soln.) | 1000.0 |
| Hydroxyethylcellulose | 15.0 |
| D.I. Water | 3023.5 |
| Flavor | 20.0 |
| Propylparaben NF | 2.0 |
| Butylparaben NF | 2.0 |
| | 5591.0 |

Place 302.35 g. of deionized water and 200 g. of 70% sorbitol solution in a 1.5 liter vessel equipped with a IKA mixer. With the agitator set at high speed add 3 g. of hydroxyethylcellulose to the water/sorbitol mixture. Continue mixing until all of hydroxyethylcellulose has dissolved. Add 307.7 g. of aluminum hydroxide gel to the vessel also under high speed agitation. Once the gel has been completely dispersed add the following ingredients in sequence; 0.4 g. butylparaben, 0.4 g. propylparaben, 4.0 g. flavor, finally add 302.35 g. of deionized water.

The suspension was then homogenized and pasteurized into plastic Bottles.

### EXAMPLE 3

Formulation Containing CaCO₃ Powder and Simethicone.

| | mg/5 ml |
|---|---|
| CaC0₃ powder | 1000.0 |
| Simethicone (30% Emulsion) | 100.0 |
| Sorbitol (70% soln.) | 1000.0 |
| Hydroxyethylcellulose | 15.0 |
| D.I. Water | 3461.0 |
| Flavor | 20.0 |
| Propylparaben NF | 2.0 |
| Butylparaben NF | 2.0 |
| | 5600.0 |

Place 500.0 g. of deionized water and 200 g. of 70% sorbitol solution in a 1.5 liter vessel equipped with a IKA mixer. With the agitator set at high speed add the CaCO3 powder. When the CaCO₃, has been completely dispersed add 3 g. of hydroxyethylcellulose to the mixture. Continue mixing until all of hydroxyethylcellulose has dissolved. Next add the 20 g. of simethicone emulsion to the vessel also under high speed agitation. Once the simethicone emulsion has been completely dispersed add the following ingredients in sequence; 0.4 g. butylparaben, 0.4 g. propylparaben, 4.0 g. flavor, finally add 192.2 g. of deionized water.

The suspension was then homogenized and pasteurized into plastic bottles.

### EXAMPLE 4

Formulation Containing Dihydroxyaluminum Sodium Carbonate (DASC).

| | Mg/5 ml |
|---|---|
| DASC | 400.0 |
| Sorbitol (70% soln.) | 1000.0 |
| Hydroxyethylcellulose | 25.0 |
| D.I. Water | 3851.5 |
| Flavor | 22.0 |
| Sodium Saccharin | 1.5 |
| | 5300.0 |

Place 385.15 g. of deionized water and 200 g. of 70% sorbitol solution in a 1.5 liter vessel equipped with a IKA mixer. With the agitator set at high speed add 5 g. of hydroxyethylcellulose to the water/sorbitol mixture. Continue mixing until all of hydroxyethylcellulose has dissolved. Add 80.0 g. of DASC to the vessel also under high speed agitation. Once the DASC has been completely dispersed add the following ingredients in sequence; 4.4 g. flavor and 0.3 g. sodium saccharin, finally add 385.15 g. of deionized water.

The suspension was then homogenized and pasteurized into plastic bottles.

### EXAMPLE 5

Formulation Containing Simethicone.

| | mg/0.6 ml |
|---|---|
| Simethicone (30% Emulsion) | 140.48 |
| Microcrystalline cellulose & | 0.60 |
| Sodium CMC | |
| Maltitol | 60.0 |
| Hydroxyethylcellulose | 1.50 |
| D.I. Water | 418.54 |
| Citric Acid | 0.75 |
| Sodium Citrate | 0.30 |
| Sodium Benzoate | 0.60 |
| Flavor | 1.20 |
| Color | 0.03 |
| | 624.00 |

Place 792.0 Kg. of deionized water to a 2000 liter tank and add 1.136 Kg. of Microcrystalline cellulose. Mix for 5 mins. before adding 2.839 Kg. of HEC, and continue mixing for a further 5 mins. Add the listed ingredients to the batch tank in the following order Citric Acid (1.42 Kg.); Sodium Citrate (0.568 Kg.); Maltitol (113.6 Kg.) and Sodium Benzoate (1.136 Kg.). Next add the Simethicone emulsion (266 Kg.) to the vessel with good agitation. Once the simethicone emulsion has been completely dispersed add the following separately prepared mixture; 57 g. of Colorant and 1.2 Kg. flavor dispersed in 1 OKg. of deionized water. Once this mixture has been added to the batch tank continue mixing for a further 30 mins.

The suspension was then homogenized and pasteurized into plastic bottles.

We claim:

1. A pasteurized heat stable liquid antacid and/or antigas preparation comprising 200 mg-2000 mg/5 ml of an aluminum-containing acid neutralizing compound and/or 20-125 mg/5 ml antigas compound in an aqueous liquid suspension containing a preservative component in an amount that does not exceed 0.05% w/v and a suspending agent consisting essentially of hydroxyethylcellulose in an amount of from 1 mg-100 mg/5 ml and, optionally, one or more other pharmaceutically acceptable additives, said preparation having a viscosity of about 100 to 1000 cps at 25° C. and 70° C.

2. A liquid antacid preparation according to claim 1, wherein the acid neutralizing compound is selected from calcium carbonate, dihydroxyaluminum sodium carbonate, magnesium carbonate, magnesium trisilicate, aluminum hydroxide and magnesium hydroxide and mixtures thereof.

3. A liquid antacid preparation according to claim 1 wherein the acid neutralizing compound is calcium carbonate.

4. A liquid antacid preparation according to claim 1 wherein the acid neutralizing compound is aluminum hydroxide gel.

5. A liquid antacid preparation according to claim 1 wherein the acid neutralizing compound is dihydroxyaluminum sodium carbonate.

6. A liquid antacid and/or antigas preparation according to claim 1 further comprising a preservative.

7. A liquid antacid and/or antigas preparation according to claim 6 wherein the preservative is selected from butylparaben, methylparaben and propylparaben.

8. A liquid antacid and/or antigas preparation according to claim 1 further comprising an antimicrobial adjuvant selected from the group consisting of propylene glycol in an amount greater than 2 weight percent and less than 15 weight percent, glycerin in an amount from about 15 to about 20 weight percent, and combinations of propylene glycol in an amount from about 3 to about 10 weight percent with glycerin in an amount from about 3 to about 10 weight percent based on the total weight of the preparation.

9. A liquid antigas preparation according to claim 1 containing about 20 mg to about 500 mg of simethicone per 5 ml.

10. A liquid antacid and/or antigas preparation according to claim 1, containing 1-100 mg/5 ml hydroxyethylcellulose.

11. A liquid antacid preparation according to claim 1, further containing a pharmaceutically effective amount of a histamine H2 receptor antagonist.

12. A liquid antacid preparation according to claim 11, wherein the histamine H2 receptor antagonist is selected from cimetidine, ranitidine, nizatidine and famotidine.

13. A liquid antacid preparation according to claim 12, containing 100 mg to about 400 mg of cimetidine per dose.

14. A liquid antacid preparation according to claim 12, containing 50 mg to about 150 mg of ranitidine per dose.

15. A liquid antacid preparation according to claim 12, containing 5 mg to 40 mg of famotidine per 5 ml.

16. A method for the treatment of a gastrointestinal disorder in a human in which acid neutralization and/or antigas treatment is desired which comprises administering to said human an effective amount of a liquid antacid and/or antigas composition of claim 1.

17. A method for the treatment of a gastrointestinal disorder in a human in which acid neutralization is desired which comprises administering to said human an effective amount of a liquid antacid composition of claim 11.

18. A method according to claim 16 wherein the gastrointestinal disorder is selected from the group consisting of acid indigestion, heartburn, dyspepsia, sour stomach, and reflux esophagitis.

19. In a method for the terminal sterilization by pasteurization in the temperature range of 60-100° C., of liquid antacid and /or antigas preparations containing a suspending agent, the improvement which comprises increasing the flow rate by using hydroxyethylcellulose as the suspending agent.

20. A pasteurized heat stable liquid antacid and/or antigas preparation comprising 200 mg-2000 mg/5 ml of an aluminum-containing acid neutralizing compound and/or 20-125 mg/5 ml antigas compound in an aqueous liquid suspension containing a preservative component in an amount that does not exceed 0.05% w/v and a suspending agent capable of being pasteurized without gelling, said preparation having a viscosity of about 100 to 1000 cps at 25° C. and 70° C.

21. A method for preparing a liquid antacid and/or antigas preparation comprising providing the liquid antacid and/or antigas preparation containing a suspending agent, homogenizing the liquid antacid and/or antigas preparation and pasteurizing the liquid antacid and/or antigas preparation in a temperature range of 60-100° C., wherein the suspending agent is hydroxyethylcellulose, and after said pasteurizing said preparation has a viscosity of about 100 to 1000 cps at 25° C. and 70° C.
